(19) United States
(12) Patent Application Publication
(10) Pub. No.: US 2010/0106202 A1
(43) Pub. Date: Apr. 29, 2010

(54) System and method for distal radioulnar joint resurfacing with dynamic fixation

(76) Inventors: Jamy Gannoe, West Milford, NJ (US); Luis A. Arellano, Fair Lawn, NJ (US)

Correspondence Address: WARD & OLIVO, SUITE 300, 382 SPRINGFIELD AVENUE, SUMMIT, NJ, 07901, US

(21) Appl. No.: 12/462,849

(22) Filed: Aug. 11, 2009

Related U.S. Application Data
(60) Provisional application No. 61/188,758, filed on Aug. 12, 2008.

(51) Int. Cl. A61B 17/56; A61F 2/42

(52) U.S. Cl. 606/86 R; 623/21.12

(57) ABSTRACT

A joint resurfacing system for repairing defects in the articular surface of the distal radioulnar joint. The system includes a hemiarthroplasty implant device for the ulna resulting in minimal removal of the underlying bone. The implant device includes a curved body, an interior surface and an opposed exterior surface, where the interior surface is coupled to an articular surface of an ulna bone. The system also includes a fixation device having a threaded end with the threaded end coupled to the implant device in order to apply a compressive force on the implant device. the fixation device may be selected from either a dynamic fixation device or a static screw device.

### CROSS-REFERENCE TO RELATED APPLICATIONS

This application claims the benefit of Provisional Application No. 61/188,758, filed Aug. 12, 2008, the entire contents of which are herein incorporated by reference.

### FIELD OF THE INVENTION

This invention relates to the field of joint prosthesis and, more particularly, to a joint resurfacing device and system for repairing defects in the distal radioulnar joint in the wrist.

### BACKGROUND OF THE INVENTION

The ulna is located at the medial side of the forearm, parallel with the radius. The ulna articulates with the radius through a pivot joint (called the distal radioulnar joint) formed between the head of the ulna and the sigmoid notch on the distal radius. The distal radioulnar joint acts as a pivot for the forearm bones. Cartilage and bone deterioration of the distal radioulnar joint is often caused by disease such as osteoarthritis, rheumatoid arthritis, trauma or other medical conditions. These medical conditions cause pain, discomfort and decrease in the range of motion in the distal radioulnar joint. Common procedures used to correct such deficiencies in the radioulnar joint include arthroplasty, arthrodesis and hemiarthroplasty. Generally, hemiarthroplasty is performed when only a portion of the native articulating surface is damaged. This procedure involves the removal of a proportional section of the damaged tissue, which is replaced with an implant. The implant serves as the new articulating surface and beneficially leaves most of the native cartilage intact. However, this procedure is currently limited within the human wrist due to the amount of bone that is required to be removed to make space for the implant. In addition, the means of fixating the implant is a well-documented issue as it is difficult to keep the implant fixed to the bone where loosening issues have been prevalent.

Currently, there is no true resurfacing approach that preserves much of the distal radioulnar joint intact and that can be installed with minimal invasive procedure. In the past, joint surface replacement included a prosthesis that comprised of two opposing parts, which were designed to be inserted into the radius and the ulna. As such, the opposing parts cooperate to provide a concave surface that pivots across a convex surface. The relative movement of the two parts against each other facilitates the natural rotating motion of the wrist. Yet, while this prosthesis may resurface the distal radioulnar joint, the resection required is significant and technically demanding and this resection limits revision options.

Another apparatus utilizes screws coupled to the ulna head. In this procedure, ligaments that connect the ulnar tip to the radius and carpus are maintained while a portion of the articular surface of the ulna is excised. A threaded screw is inserted into the excised articular surface and the procedure is finished by attaching a cylindrical cap to the screw. The cylindrical cap forms the new articular surface of the ulna and contacts the radius during natural movement of the distal radioulnar joint. However, this procedure is invasive as it requires significant cartilage and bone removal.

There is therefore a need for a joint resurfacing device, system and method of use that overcomes some or all of the previously delineated drawbacks of prior joint resurfacing procedures and devices.

### SUMMARY OF THE INVENTION

An object of the invention is to overcome the drawbacks of previous inventions.

Another object of the invention is to provide a novel and useful joint resurfacing device for restoring the articulating surface of the ulna through a minimally invasive procedure.

Another object of the invention is to provide a system for restoring the articulating surface of the ulna with a dynamic fixation assembly.

Another object of the invention is to provide a method for resurfacing the articulating surface of the ulna.

Another object of the invention is to provide a method that preserves as much native bone to allow more options for revision surgery.

In a first non-limiting aspect of the invention,

An assembly for joint resurfacing is provided and includes an implant device. The implant device includes a curved body, an interior surface and an opposed exterior surface, where the interior surface is coupled to an articular surface of an ulna bone. The assembly also includes a fixation device having a threaded end, where the fixation device is coupled to the implant device at the threaded end, thereby applying a compressive force on the implant device. In some non-limiting embodiments, the fixation device may be selected from either a dynamic fixation device or a static screw device.

In a second non-limiting aspect of the invention, a system for resurfacing the distal radioulnar joint in a wrist is provided. The system includes an implant device for attaching to an ulna bone, where the implant device has a curved body, an interior surface and an opposed exterior surface. The system also includes a fixation deice and an implant holder assembly. The fixation device has a threaded end for applying a dynamic compressive force on the implant device, where the fixation device is coupled to the implant device at the threaded end. The implant holder assembly further includes an elongated member having a slot at a third end, a first protrusion at a fourth end, and a longitudinal aperture traversing a length of the elongated member; an elongated rod coupled to the longitudinal aperture for grasping the implant device; a modular arm having a rail at a fifth end and a third aperture at a sixth end, where the rail is slideably coupled to the slot; and a guide barrel coupled to the third aperture.

In a third non-limiting aspect of the invention, a method for resurfacing a distal radioulnar joint is provided. First, an incision is made in a wrist to expose the distal radioulnar joint. Next, retractors are utilized to slightly elevate the ulna in order to ensure that there is an adequate working area. Next, a trial inserter device is used to assist the surgeon in achieving the proper depth and alignment of implant device. Next, the implant device is coupled to implant holder assembly by aligning feet on the implant holder assembly with the slots in the implant device. Next, the surgeon may position implant holder assembly together with the implant device onto the ulna in order to locate the correct and desired position of the implantation site. Next, a modular arm is coupled to the implant holder assembly by aligning the rail and slot features of the modular arm and the implant holder assembly. The guide barrel is then inserted into the corresponding hole on the modular arm and tightened onto the ulna. Next, a hole and trajectory is prepared with proper alignment for a dynamic fixation device. Next, guide barrel is removed and a dynamic fixation device is inserted using the hole as an alignment aid. As dynamic fixation device connects to implant device, the implant device begins tensioning. Once a predetermined torque level has been reached, the spring element contained within dynamic fixation device begins to stretch applying dynamic compression.

### BRIEF DESCRIPTION OF THE DRAWINGS

A further understanding of the invention may be obtained by reference to a preferred embodiment set forth in the illustrations of the accompanying drawings. Although the illustrated embodiment is merely exemplary of systems and methods for carrying out the invention, both the organization and method of operation of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the invention, reference is now made to the following drawings in which:

FIG. 1 is a perspective view of an implant device used in the joint resurfacing system in accordance with a preferred embodiment of the invention.

FIG. 2 is a perspective view of the implant device used in the joint resurfacing system shown in FIG. 1 according to the preferred embodiment of the invention.

FIG. 3 is a side view of the implant device shown in FIG. 2 according to the preferred embodiment of the invention.

FIG. 4 is a bottom view of the implant device shown in FIG. 2 according to the preferred embodiment of the invention.

FIG. 5 is a perspective view of the implant holder and placement assembly of the joint resurfacing system according to the preferred embodiment of the invention.

FIG. 6 is a perspective view of the trial inserter device according to the preferred embodiment of the invention.

FIG. 7A illustrates a step of installing the implant device of FIG. 2 according to the preferred embodiment of the invention.

FIG. 7B illustrates another step of installing the implant device of FIG. 2 using the trial inserter device of FIG. 6 according to the preferred embodiment of the invention.

FIG. 7C illustrates another step of installing the implant device of FIG. 2 using an implant holder assembly shown in FIG. 5 according to the preferred embodiment of the invention.

FIG. 7D illustrates another step of installing the implant device of FIG. 2 using an implant holder and placement assembly shown in FIG. 5 according to the preferred embodiment of the invention.

FIG. 7E illustrates another step of installing the implant device of FIG. 2 and the implant holder and placement assembly of FIG. 5 according to the preferred embodiment of the invention.

FIG. 7F illustrates another step of installing the implant device of FIG. 2 using the dynamic fixation device of FIG. 1 and the implant holder and placement assembly of FIG. 5 according to the preferred embodiment of the invention.

FIG. 8 is a flow chart illustrating the method of coupling the implant device of FIG. 2 to an ulnar bone according to the preferred embodiment of the invention.

FIG. 9 is a perspective view of an implant device used in a joint resurfacing system in accordance with an alternate embodiment of the invention.

### DETAILED DESCRIPTION OF THE EMBODIMENTS OF THE INVENTION

The invention may be understood more readily by reference to the following detailed description of preferred embodiment of the invention. However, techniques, systems and operating structures in accordance with the invention may be embodied in a wide variety of forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

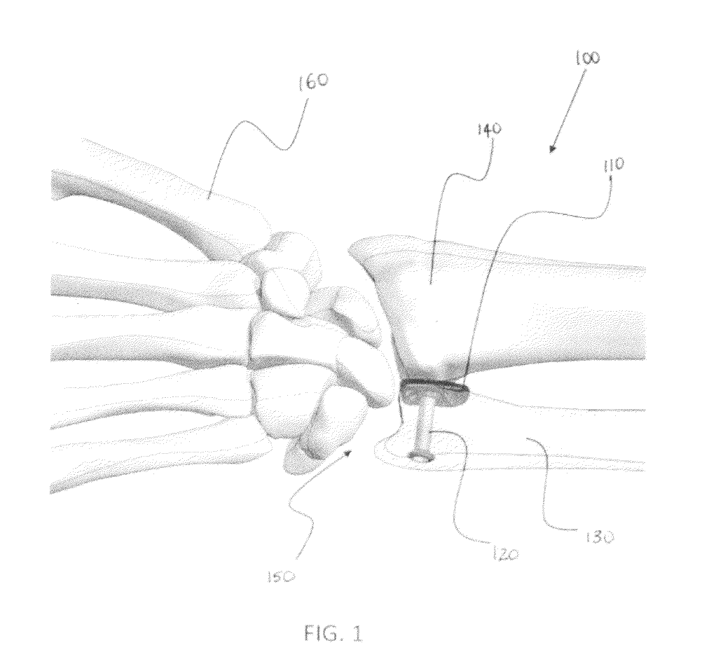

Referring now to FIG. 1, there is shown a joint resurfacing system 100 in accordance with the teachings of the preferred embodiment of the invention. As shown, joint resurfacing system 100 includes an implant device or implant 110 selectively coupled to a dynamic fixation device 120. Ulna bone 130 meets radius bone 140 at a distal end 150 and at a proximal end (not shown). Implant 110 is coupled to ulna bone 130 at distal end 150 in order to replace any deteriorated or damaged bone or tissue. Dynamic fixation device 120, being selectively coupled to implant 110, applies a dynamic compressive force on implant 110, thereby securely coupling implant 110 to the articulating surface of ulna 130. It should be appreciated that implant 110 is used to perform hemiarthroplasty using a minimal invasive procedure for restoration of the articulating surface of ulna 130. In other non-limiting embodiments, implant 110 may vary in size in order to resurface only part of articulating surface of ulna 130.

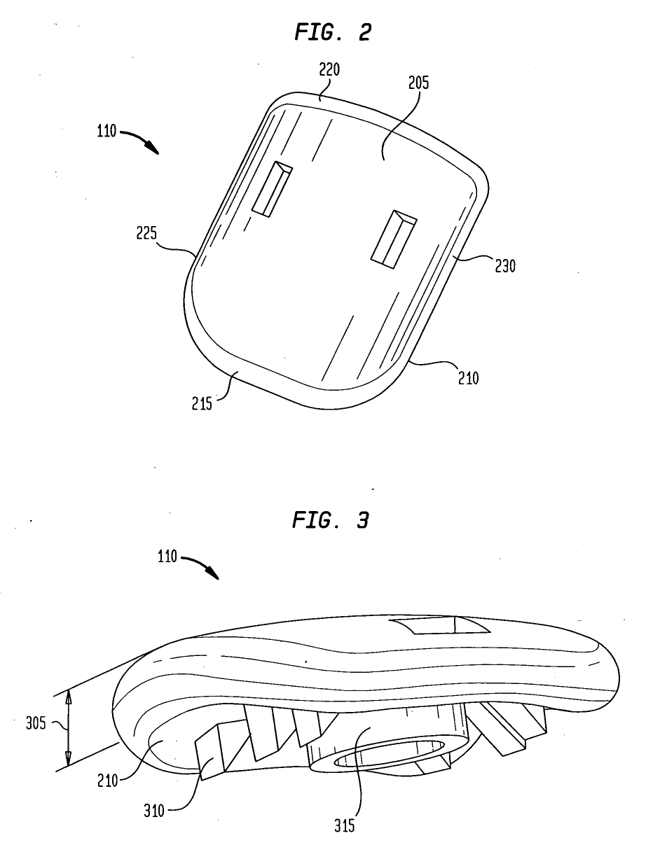

As shown in FIGS. 2, 3, and 4, implant 110 is generally curved in shape (i.e., implant 110 has a generally “potato-chip” shape). Particularly, implant 110 is anatomical in shape with curvatures to match the prepared bone surface and the existing contours of ulna 130. As shown in FIG. 2, implant 110 has a superior surface 205 and an opposed interior surface 210. Superior surface 205 is the radial face that provides the articulating surface for radius 140. Interior surface 210 is the ulnar face that will be affixed onto the prepared ulna 130. Implant 110 further comprises a first proximal edge 215, an opposed distal edge 220, a left edge 225 and a right edge 230. Implant 110 is designed to mimic the native tissue as well as the tomography of the native healthy ulna 130.

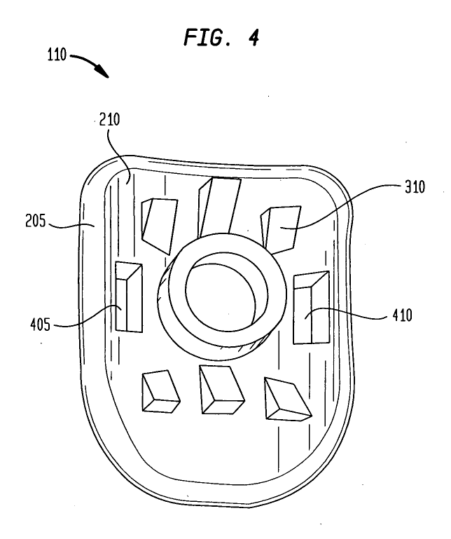

As shown in FIG. 3, implant 110 has a low profile to minimize distraction required for implantation and facilitate a tissue sparing technique. Preferably, implant 110 comprises a thickness 305, having a range of 0.5-4 mm. It should also be appreciated that thickness 310 of implant 110 provides beneficial properties resulting in a lower stress shielding while reducing the amount of removed native tissue. This reduction in removal of native tissue reduces the violation of the distal radioulnar joint. Also, implant 110 has a plurality of substantially similar keels or holding spikes 310, which are formed on interior surface 210. Holding spikes 310 comprise tapered edges provided to cause holding spikes 310 to travel through the bone material as implant 110 couples to the bone surface. As such, holding spikes 310 provide additional primary stability to implant 110. Implant 110 also comprises a threaded tubular post 315 provided on surface 210. Post 315 has a plurality of substantially similar internal threads (not shown) provided to receive a complementary threaded end of a dynamic fixation device 120 to secure implant 110 to ulna bone 130.

It should be appreciated that in one non-limiting embodiment, implant 110 may be made from a Titanium material, although, in other non-limiting embodiments, implant 110 may be made from Cobalt Chrome, Stainless Steel, PEEK, PE, NiTi or any other biocompatible material. It should also be appreciated that interior surface 210 may be coated with a porous undercoating such as plasma coated Titanium, mesh Titanium, or other similar types of osteoconductive materials, in order to provide an osteoconductive surface for fixating implant 110 to the ulnar surface. In other non-limiting embodiments, surface 210 may be infused with hydroxylapatite or other similar types of materials to increase connection to the surface of ulna 130.

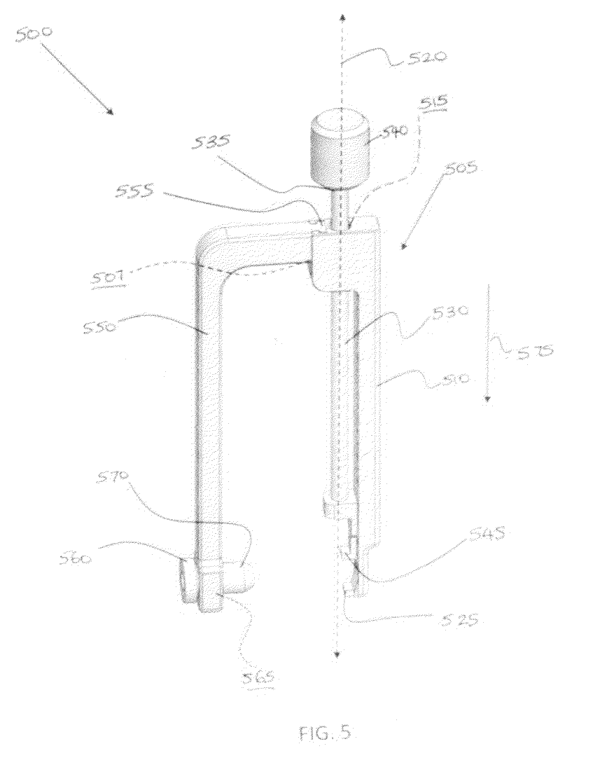

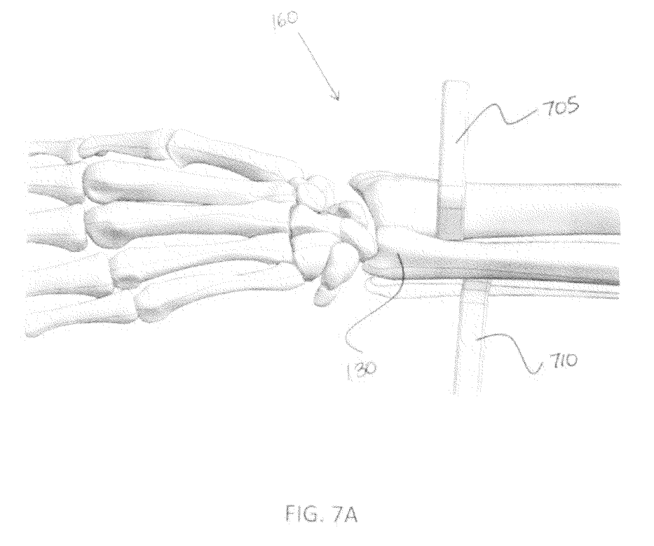

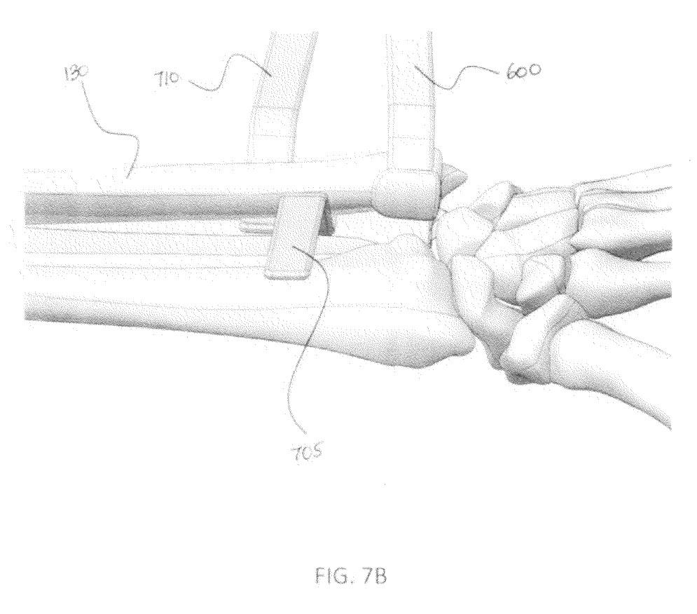

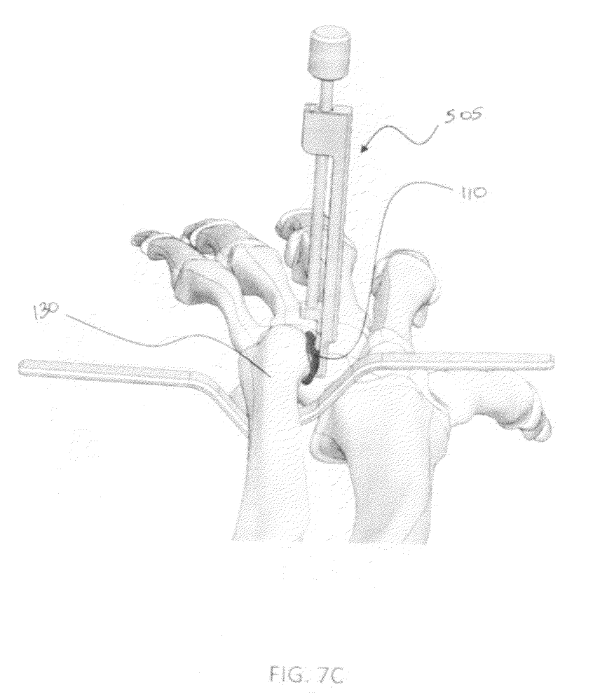

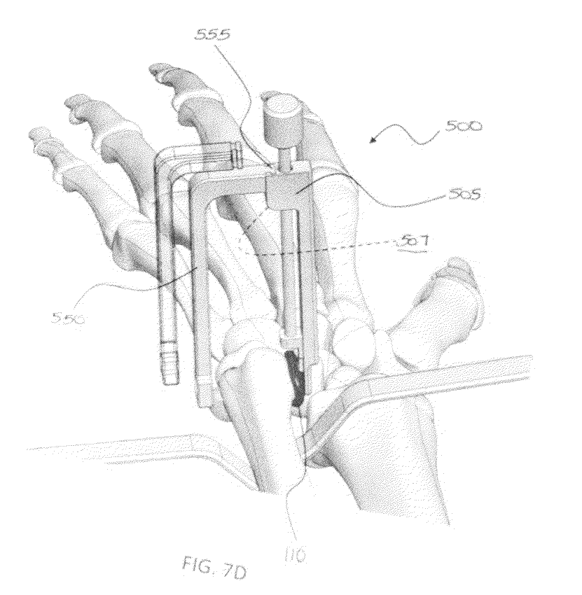

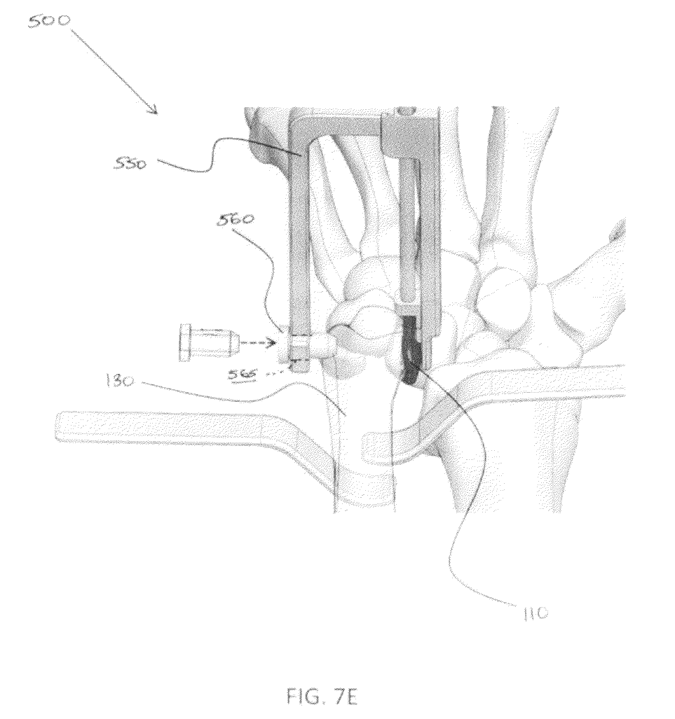

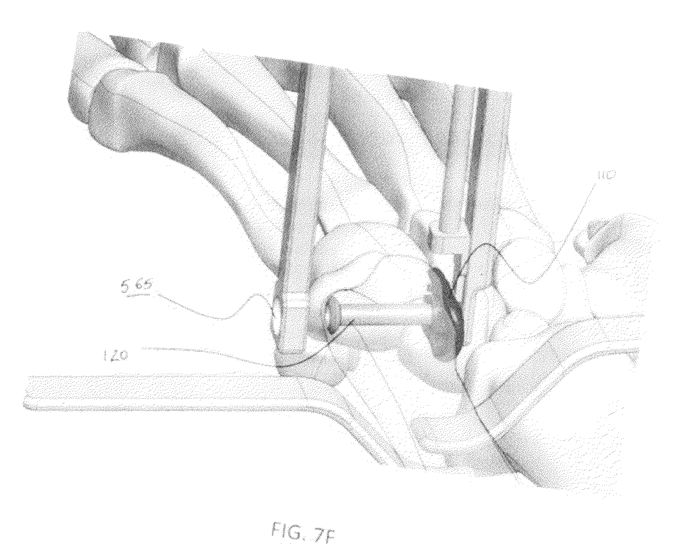

As shown in FIG. 4, implant 110 has a plurality of substantially similar slots 405 and 410. Slots 405 and 410 are provided on body of implant 110 and traverse from superior surface 205 to interior surface 210. Slots 405 and 410 are provided to receive an implant holder and placement tool in order to position implant 110 on the articulating surface of ulna 130 (as shown in FIGS. 7C-7F).

FIG. 5 shows an implant holder and placement assembly 500 (also called “implant holder assembly 500”) for grasping implant 110 for insertion into ulna 130. Particularly, implant holder assembly 500 includes an implant holder device 505 comprising a generally “L-shaped” rigid portion 510. Portion 510 has a slot 507 at one end that terminates into a rigid protrusion or foot 525 at an opposed end. Also, portion 510 has an aperture 515 aligned along longitudinal axis 520. Further, implant holder device 505 has an elongated rod 530 with first end 535 terminating into a cylindrical knob 540 and a second end terminating into a rigid protrusion or foot 545. Rod 530 is aligned along longitudinal axis 520, with rod 530 being contained within aperture 515. Turning knob 540 in a clockwise direction causes foot 545 to travel along longitudinal axis 540, thereby increasing or decreasing the distance of foot 545 relative to foot 525. Thus, the distance between feet 545 and 525 may either be increased or decreased by turning the knob 540.

Implant holder assembly 500 further comprises modular arm 550 coupled to implant holder device 505 through a rail and slot system. Particularly, modular arm 550 is generally “L-shaped” with a rail 555 provided at one end and an aperture 565 at second end. Rail 555 is generally “T-shaped” and is provided to be received in complementary slot 507. Modular arm 550 is slidably coupled to implant holder device 505 by inserting rail 555 into slot 507 and sliding rail 555 in direction 575 until rail 555 is securely and frictionally held in place by slot 507. Aperture 565 receives a generally tubular guide barrel 560 through a threaded connection. Particularly, the interior surface of aperture 565 has threads (not shown) that are complementary to exterior threaded surface 570 of guide barrel 560. Guide barrel 560 has a longitudinal aperture (not shown) which, in one non-limiting embodiment, acts as a guide for receiving a drill. In other non-limiting embodiments, guide barrel 560 may act as a guide for wire placement, taps and screw insertion.

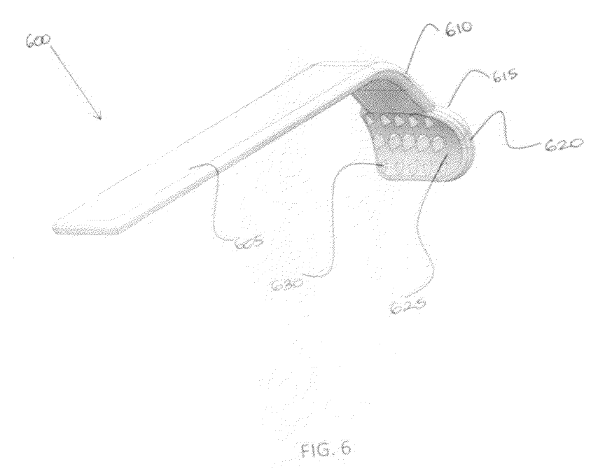

FIG. 6 illustrates a trial inserter device 600 for preparing ulna 130 for insertion of implant 110. Particularly, trial inserter device 600 has an elongated and planar rod portion 605 coupled to a generally curved portion 610. Further, curved portion 610 terminates into a generally curved trial device 615 that is substantially similar to implant device 110. Trial device 615 comprises a first exterior surface 620 and an interior surface 625. Interior surface 625 may have a plurality of cutting elements to allow a rasp function for preparing the surface for the implant. Trial-inserter device 600 is utilized to assist the surgeon in achieving the proper depth, positioning and alignment of implant 110. Also, trial inserter device 600 assists the surgeon in estimating the size of an implant that is needed for distal radioulnar joint resurfacing, as will be later shown and described.

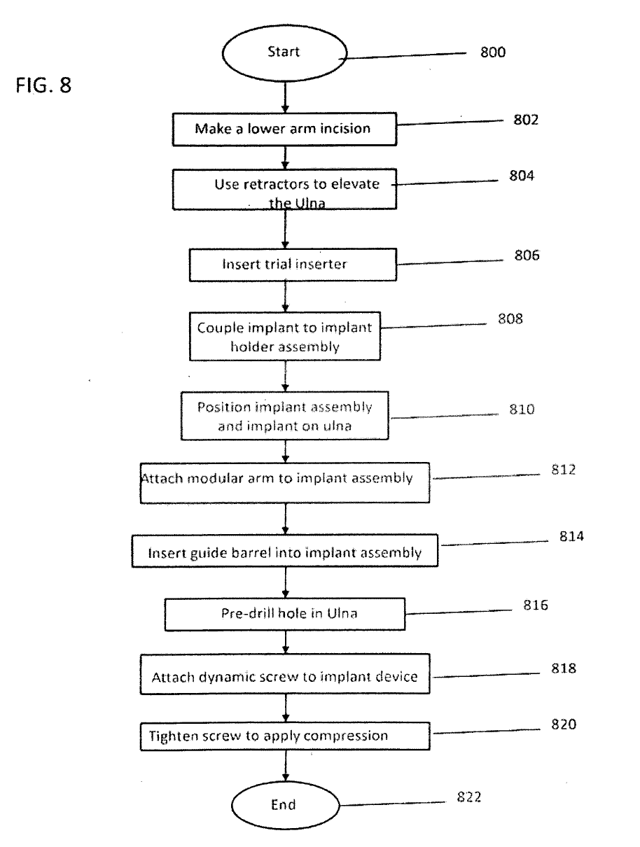

In operation, and as best shown in FIGS. 7A-7F and 8, implant device 110, implant holder assembly 500 and trial inserter device 600 may be utilized to provide a system for resurfacing an articulating surface of ulna 130 in a human arm 160. The method starts in step 800 and proceeds to step 802, whereby a lower-arm incision is made in arm 160 to expose the distal radioulnar joint and specifically ulna 130. The distal radioulnar joint is exposed to remove damaged tissue, which may include the removal of at least the bone or tissue from the articulating surface that has been deteriorated or damaged. Next, in step 804, as shown in FIG. 7A, retractors 705 and 710 are utilized to slightly elevate ulna 130 in order to ensure that there is an adequate working area. Next, in step 806 and as shown in FIG. 7B, a trial inserter device 600 is used to assist the surgeon in achieving the proper depth and alignment of implant device 110. Trial inserter device 600 also allows the surgeon to determine the size of implant device 110 that would best fit the articulating surface of ulna 130. Trial inserter device 600 is placed firmly against ulna 130 while ulna 130 is returned to its neutral position. This aids in further examining the sizing as well as articulation with the sigmoid notch.

Once the proper implant device 110 is obtained, in step 808 implant device 110 is coupled to implant holder assembly 500 (shown in FIG. 5). Implant holder assembly 500 is coupled to implant device 110 by aligning feet 525 and 545 with slots 405 and 410 (shown in FIG. 4). Turning knob 540 of implant holder assembly 500 allows feet 525 and 545 to translate in order to match the corresponding slot positions on implant device 110. Once implant device 110 is firmly and accurately placed onto implant holder assembly 500, knob 540 is rotated to place either tension or compression on implant device 110. The tension or compression provides the force needed to rigidly fix and hold implant device 110 during implantation. Next in step 810, as shown in FIG. 7C, the surgeon will position implant holder assembly 500 together with implant device 110 onto ulna 130 in order to locate the correct and desired position of the implantation site. Once the site has been established, the site is prepared for implantation by minor burring or rasping of the articulating surface. Burring down or rasping of ulna bone 130 will aid in implant fixation, placement and bone on-growth. This also aids in having a smooth transition between the existing ulna bone 130 and implant device 110.

Next, in step 812, as shown in FIG. 7D, modular arm 550 is coupled to the implant holder assembly 500 by aligning rail 555 of modular arm 550 and slot 507 features of implant holder assembly 500. The “T-shaped” rail 555 and complementary “T-shaped” slot 507 aid in the proper stability and alignment of implant holder assembly 500 during the surgical procedure. Next, in step 814 and as shown in FIG. 7E, guide barrel 560 is inserted into the corresponding hole 565 on modular arm 550. Once aligned, guide barrel 560 is tightened onto ulna 130 by turning guide barrel 560. The force achieved by tightening guide barrel 560 onto ulna 130 allows implant holder assembly 500 to act like a clamp and maintain the desired implant device 110 position relative to the ulna 130. Guide barrel 560 also allows the surgeon to visualize the placement of a guide wire (not shown), such as a Kirschner wire, drills, taps and screws through a posterior stab incision. In step 816, a hole and trajectory is prepared with proper alignment in ulna 130 for a dynamic fixation device 120.

Next in step 818 and as shown in FIG. 7F, guide barrel 560 is removed and a dynamic fixation device 120 is inserted using hole 565 as an alignment aid. In another non-limiting embodiment, guide barrel 560 may be utilized as an alignment aid for insertion of dynamic fixation device 120. Specifically, dynamic fixation device 120 comprises a first member positioned at a proximal end, a second threaded member positioned at a distal end and a tubular dynamic element coupled to first member and second threaded member. The tubular dynamic element cooperates with first and second members to exert a dynamic compressive force. The threaded dynamic element may be stretched, causing the first member and second threaded member to be in dynamic compression. In other non-limiting embodiments, any other type of screw device may be utilized. In step 820, as dynamic fixation device 120 connects to implant device 110, implant device 110 begins tensioning. Once a predetermined torque level has been reached, the tubular dynamic element (not shown) contained within dynamic fixation device 120 begins to stretch applying dynamic compression. This compression is maintained over a longer period of time as compared to normal static fixation devices. The method ends in step 822. It should be appreciated that FIGS. 7A-7F show the fixation of implant device 110 in ulna 130 bone of the distal radioulnar joint. However, in other non-limiting embodiments, alternate similar approach may be utilized to install implant device 110 on radius 140.

The invention provides an implant device 110 for ulna resurfacing resulting in minimal removal of the underlying bone. Implant device 110 and the aforementioned instrumentation allow for a tissue sparing technique and minimize bone carpentry. It also avoids damage to the critical anatomy such as the ulnar nerve or the Triangular Fibro cartilage Complex (TFCC). This approach also allows for a short recovery as well as a lesser chance of implant loosening. Importantly, implant device 110 has a thickness that lowers stress shielding and allows for minimal removal of native tissue. Implant device 110 also preserves the option of having revision surgery in the future. Thus, implant device 110 provides a faster recovery period due to minimal invasive surgery that replaces just the damaged portion of the ulnar dome.

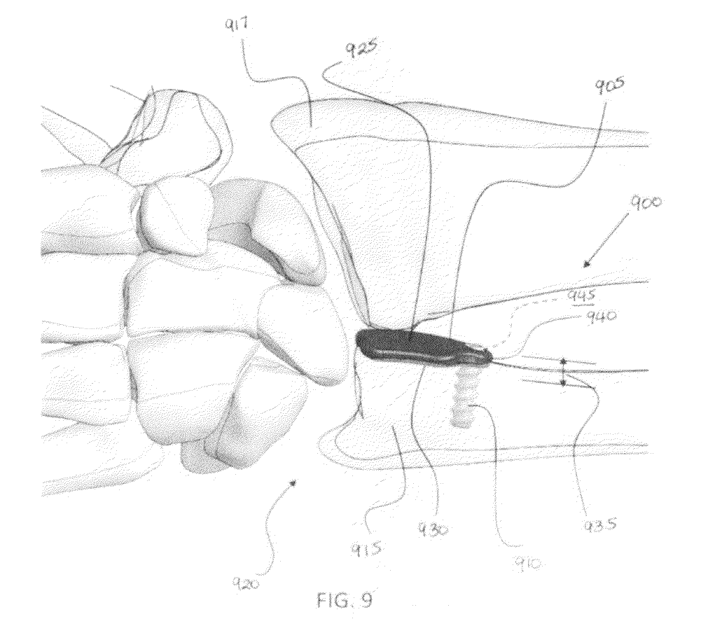

In an alternate embodiment, as shown in FIG. 9, joint resurfacing system 900 comprises an implant device 905 selectively coupled to a threaded screw 910. Further, implant device 905 is coupled to ulna bone 915 at the distal radioulnar joint in order to replace any deteriorated or damaged bone or tissue. Implant device 905 is generally curved in shape, with curvatures to match the prepared bone surface and the existing contours of the ulna 915. Implant 905 has a superior surface 925 and an opposed interior surface 930. Superior surface 925 is the radial face that provides the articulating surface for radius 917 and interior surface 930 is the ulnar face that will be affixed onto ulna 915. Implant 905 has a low profile to minimize irritation and has a thickness 935, preferably comprising a range of 0.5-4 mm. Also, implant 905 has a plurality of substantially similar keels or holding spikes formed on interior surface 930, such as holding spike 310 of the preferred embodiment that was shown in FIGS. 2-4.

Further, implant 905 has an aperture or hole 945 provided at a proximal end 940. Hole 945 receives a threaded screw 910, which traverses ulna 915, in order to couple implant 905 to ulna 915. Thus, implant 905 and screw 910 provide a system 900 for resurfacing the distal radioulnar joint by limiting the amount of native tissue and bone removed.

It should be understood that this invention is not limited to the disclosed features and other similar method and system may be utilized without departing from the spirit and the scope of the invention.

While the invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the invention is capable of being embodied in other forms without departing from its essential characteristics.

1. An assembly for joint resurfacing, comprising:
an implant device, wherein said implant device further comprising a curved body, an interior surface and an opposed exterior surface, wherein said interior surface is coupled to an articular surface of a ulna bone; and
a fixation device having a threaded end, wherein said fixation device is coupled to said implant device at said threaded end thereby applying a compressive force on said implant device.

2. The assembly of claim 1, wherein said fixation device is a dynamic compression device.

3. The assembly of claim 1, wherein said fixation device is a static screw.

4. The assembly of claim 1, wherein said exterior surface is coupled to a radial bone surface.

5. The assembly of claim 1, wherein said curved body further comprises a plurality of edges to mimic a surface of said ulna bone.

6. The assembly of claim 1, further comprising a plurality of spikes on said interior surface of said implant device.

7. The assembly of claim 6, wherein each of said plurality of spikes has a tapered edge.

8. The assembly of claim 1, wherein said interior surface is concave.

9. The assembly of claim 1, wherein said implant device further comprises a plurality of slots disposed in said curved body.

10. The assembly of claim 9, wherein said plurality of slots receive an implant tool for coupling said implant device to said ulna bone.

11. The assembly of claim 10, wherein said interior surface has a threaded aperture for receiving said threaded end of said fixation device.

12. The assembly of claim 11, wherein said threaded aperture partially traverses said curved body orthogonally to said interior surface.

13. The assembly of claim 7, wherein said plurality of spikes are tapered for traveling through bone material.

14. A system for joint resurfacing, comprising:
an implant device for attaching to a ulna bone, wherein said implant device having a curved body, an interior surface and an opposed exterior surface;
a fixation device having a threaded end for applying a compressive force on said implant device, wherein said fixation device is coupled to said implant device at said threaded end; and
an implant holder assembly, wherein said implant holder assembly further comprising:
an elongated member having a slot at a third end, a first protrusion at a fourth end, and a longitudinal aperture traversing a length of said elongated member;
an elongated rod coupled to said longitudinal aperture for grasping said implant device;
a modular arm having a rail at a fifth end and a third aperture at a sixth end, wherein said rail is slideably coupled to said slot; and
a guide barrel coupled to said third aperture.

15. The system of claim 14, wherein said fixation device is a dynamic compression device.

16. The system of claim 15, wherein said fixation device is a static screw.

17. The system of claim 14, wherein said exterior surface is coupled to a radial bone surface.

18. The system of claim 14, wherein said curved body further comprises a plurality of edges to mimic an articulating surface of said ulna bone.

19. The system of claim 14, further comprising a plurality of spikes on said interior surface of said implant device.

20. The system of claim 19, wherein each of said plurality of spikes has a tapered edge.

21. The system of claim 14, wherein said interior surface is concave.

22. The system of claim 14, wherein said implant device further comprises a plurality of slots disposed in said curved body.

23. The system of claim 22, wherein said plurality of slots receive said implant holder for coupling said implant device to said ulna bone.

24. The system of claim 23, wherein said interior surface has a threaded aperture for receiving said threaded end of said fixation device.

25. The system of claim 24, wherein said threaded aperture partially traverses said curved body orthogonally to said interior surface.

26. The system of claim 20, wherein said plurality of spikes are tapered for traveling through bone material.

27. The system of claim 22, wherein said elongated rod further comprises a cylindrical knob at a seventh end and an opposed second protrusion at an eighth end, wherein turning said knob causes said second protrusion to move relative to said first protrusion.

28. The system of claim 27, wherein said first and second protrusions are frictionally coupled to said plurality of slots.

29. A method for joint resurfacing, comprising:
making an incision and exposing a distal radioulnar joint;
distracting the radioulnar joint;
inserting a trial inserter device and selecting an implant device;
coupling an implant holder assembly to the implant device;
attaching implant holder assembly to the ulna bone and rotating a guide barrel to frictionally couple the implant device onto the ulna bone;
predrilling a hole in the ulna bone;
inserting a fixation device in the ulna bone and threadably coupling the fixation device to the implant device; and
removing the implant holder assembly.

30. The method of claim 29, further comprising utilizing the trial inserter device to achieve a proper depth and alignment of the implant device.

31. The method of claim 30, further comprising slideably coupling a modular arm to the implant holder assembly.

32. The method of claim 29, wherein the implant device comprises a curved body, an interior surface and an opposed exterior surface.

33. The method of claim 32, wherein the fixation device comprises a threaded end for applying a compressive force on the implant device, wherein the fixation device is coupled to the implant device at the threaded end.

34. The method of claim 33, wherein the fixation device is a dynamic compression device.

35. The method of claim 33, wherein the fixation device is a static screw.

36. The method of claim 33, wherein the implant holder assembly comprises:
an elongated member having a slot at a first end, a first protrusion at a second end, and a longitudinal first aperture traversing a length of the elongated member;
an elongated rod coupled to the first aperture for grasping the implant device;
a modular arm having a rail at a third end and a second aperture at a fourth end, wherein the rail is slideably coupled to the slot; and
a guide barrel coupled to said third aperture.

37. The method of claim 32, wherein the exterior surface is coupled to a radial bone surface.

38. The method of claim 37, wherein the curved body includes a plurality of edges to mimic an articulating surface of the ulna bone.

39. The method of claim 38, wherein the curved body includes a plurality of spikes on the interior surface.

40. The method of claim 39, wherein each of the plurality of spikes has a tapered edge.

41. The method of claim 40, wherein the interior surface is concave.

42. The method of claim 41, wherein the implant device further comprises a plurality of elongated slots disposed in the curved body.

43. The method of claim 42, wherein the plurality of elongated slots receive the implant holder assembly for coupling the implant device to the ulna bone.

44. The method of claim 43, wherein the interior surface has a threaded aperture for receiving the threaded end of the fixation device.

45. The method of claim 44, wherein the threaded aperture partially traverses the curved body orthogonally to the interior surface.

46. The method of claim 40, wherein the plurality of spikes are tapered for traveling through bone material.

47. The method of claim 46, wherein the elongated rod includes a cylindrical knob at a fifth end and an opposed second protrusion at an sixth end, wherein turning the knob causes the second protrusion to move relative to the first protrusion.

48. The method of claim 47, wherein the first and second protrusions are frictionally coupled to the plurality of elongated slots.
